(19) United States
(12) Patent Application Publication
(10) Pub. No.: US 2025/0228712 A1
(43) Pub. Date: Jul. 17, 2025

(54) HIGH SHEER STRENGTH COMPRESSION WRAPS TO APPLY KNOWN PRESSURES

(71) Applicant: BioSkin, LLC, Ashland, OR (US)

(72) Inventors: Quinn Daniel Cutler, Medford, OR (US); Tanner Edward Cropper, Ashland, OR (US); Michael Edwin Munter, Ashland, OR (US)

(21) Appl. No.: 18/412,384

(22) Filed: Jan. 12, 2024

(51) Int. Cl. A61F 13/02; A61F 13/00

(52) U.S. Cl. CPC A61F 13/0273; A61F 13/00059; A61F 2013/00489; A61F 2013/00565

(57) ABSTRACT

Embodiments of the inventive subject matter are directed to non-sticky, adhesive-free wraps. Wraps disclosed in this application are designed to stick to themselves by implementing a material that comprises fibers on one side and loops on the other. The selected materials have high sheer strength and low breakaway strength, so they are easy to put on and take off and will not fall off accidentally. Wraps of the inventive subject matter additionally include regularly spaced lines that facilitates application of known magnitudes of pressure. By using a key that is tuned to a specific wrap, users can apply a desired amount of pressure to a limb.

### FIELD OF THE INVENTION

The field of the invention is compression wraps.

### BACKGROUND

Wraps can be used in many different contexts, including in wound care, physical therapy, medicine, and the like. Previous approaches for wraps have primarily focused on solutions that use adhesives, use inelastic materials, incorporate scratchy or rough materials and so on. These approaches typically involve materials like tape and hook and loop solutions such as Velcro. Some other solutions that can be used to apply compression to a particular part of the body include compression sleeves, compression socks and so on. However, these approaches have several limitations. For instance, materials that incorporate adhesives can leave sticky residue and can painfully tug on body hair upon removal. Inelastic materials can make it difficult to apply light compressive pressure. And compression sleeves and the like will apply different pressures to different people dependent on size.

All these possible solutions also fail to facilitate application of known, consistent pressures to a person's body part, while using advanced materials that make consistent and comfortable application of the wrap possible while also making multiple reapplications of the wrap feasible without degrading the wrap.

### SUMMARY OF THE INVENTION

The present invention provides systems and methods directed to wraps used in medicine, sports, and wound care. In one aspect of the inventive subject matter, a compression wrap system is contemplated, comprising: an elongated fabric material having a first side, a second side, and a series of equally spaced lines across the elongated fabric; the first side comprising loops and the second side comprising fibers, where the first side is configured to stick to the second side; a key having a first line and a second line; and where, when using the elongated fabric as a wrap, aligning the first line of the key with a first equally spaced line on the elongated fabric and stretching the elongated fabric such that a second equally spaced line is aligned with the second line of the key results in a known pressure being applied by the wrap.

In some embodiments, the key has an edge, and the edge coincides with the first line. The loops of a wrap can have a linear mass density of between 0.04 deniers (D) and 0.15 D, and the loops on the first side can a density of between 1,000,000 and 1,500,000 loops per square inch. The fibers can have a linear mass density of between 1 D and 5 D, and the fibers on the second side can have a density of between 90,000 and 110,000 fibers per square inch.

In another aspect of the inventive subject matter, a compression wrap system is contemplated, comprising: a wrap comprising an elongated fabric material; the wrap having a first side, a second side, and a series of equally spaced lines across a width of the wrap; the first side comprising loops and the second side comprising fibers, where the loops of the first side are configured to stick to the fibers of the second side, and where, upon applying a fixed amount of stretch to the wrap when the wrap is wrapped around an object, a known amount of pressure is applied; and where the fixed amount of stretch is measured using the equally spaced lines.

In some embodiments, the wrap system also includes a key having a first line and a second line, where the key is used to measure the fixed amount of stretch by aligning the first line with a first equally spaced line and the second line with a second equally spaced line. The key can include an edge, and the edge can coincide with the first line. The loops of a wrap can have a linear mass density of between 0.04 deniers (D) and 0.15 D, and the loops on the first side can a density of between 1,000,000 and 1,500,000 loops per square inch. The fibers can have a linear mass density of between 1 D and 5 D, and the fibers on the second side can have a density of between 90,000 and 110,000 fibers per square inch.

One should appreciate that the disclosed subject matter provides many advantageous technical effects including predictability in applying pressure when applying a wrap, while maintaining ease of use and convenience.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWING

FIG. 1 shows a wrap of the inventive subject matter.

FIG. 2 shows a wrap being applied to user's lower leg.

FIG. 3 shows a key that can be used with a wrap.

FIG. 4 shows a key in use with a wrap in an unstretched state.

FIG. 5 shows a key in use with a wrap in a first stretched state.

FIG. 6 shows a key in use with a wrap in a second stretched state.

FIG. 7 shows an example of a wrap having an EVA core.

FIG. 8 shows an example of a wrap having a PU core.

FIG. 9 shows an example of a wrap having a TPU core.

### DETAILED DESCRIPTION

The following discussion provides example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

As used in the description in this application and throughout the claims that follow, the meaning of “a,” “an,” and “the” includes plural reference unless the context clearly dictates otherwise. Also, as used in the description in this application, the meaning of “in” includes “in” and “on” unless the context clearly dictates otherwise.

Also, as used in this application, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Moreover, and unless the context dictates the contrary, all ranges set forth in this application should be interpreted as being inclusive of their endpoints and open-ended ranges should be interpreted to include only commercially practical values. Similarly, all lists of values should be considered as inclusive of intermediate values unless the context indicates the contrary.

Systems and methods of the inventive subject matter are directed to compression wraps that are designed to apply known pressures when stretched according to an accompanying key. Materials used to make the wrap are designed to have high sheer strength when stuck to itself. For example, when an embodiment of the inventive subject matter is wrapped around a user's leg, the top surface of the wrap comes into contact with its own bottom surface as the wrap goes around the user's leg, and the contact between the top surface of the bottom surface has high sheer strength.

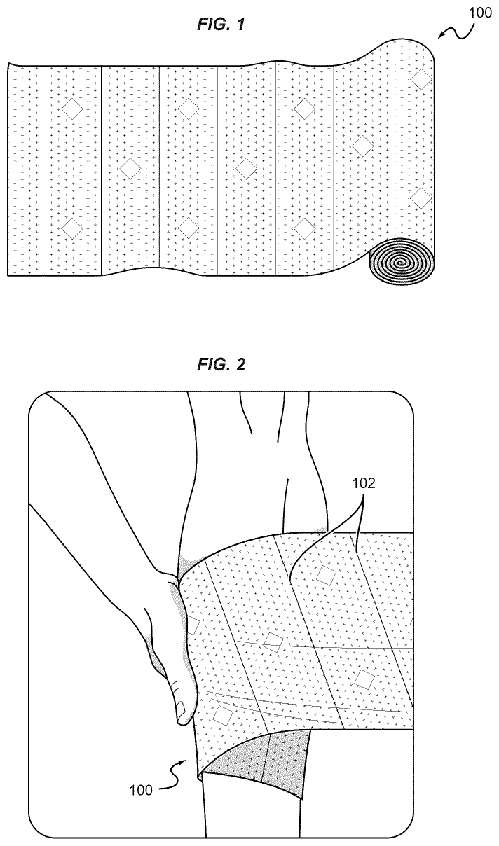

FIG. 1 shows a wrap 100 of the inventive subject matter having lines 102 marked on it at regular intervals. A wrap of the inventive subject matter is made from a non-adhesive sticky fabric. Non-adhesive sticky fabrics are better than many alternative materials because they are easily reusable. Ordinary adhesives lose their adhesive abilities after a low number of cycles, whereas sticky fabrics of the inventive subject matter are reusable for a much larger number of cycles, making adhesive tapes inappropriate substitutes. Wrap materials of the inventive subject matter are made from non-adhesive sticky fabric will be referred to as a “wrap” in this application.

Non-adhesive sticky wraps of the inventive subject matter can feature fibers on one side and loops on the other. Thus, when a loop side contacts a fiber side, the loops couple with the fibers to create the sticky property that has high enough sheer strength to hold the wrap onto itself while in use. Fibers are measured in denier units. Denier (or “den,” abbreviated D), is a unit of measure for the linear mass density of fibers that measures mass in grams per 9,000 meters of a fiber. Wraps of the inventive subject matter should be made using fibers that have between 1 D and 5 D (preferably around 3.125 D) linear mass density. Loops implemented into wraps of the inventive subject matter should have linear mass densities ranging from of 0.04 D to 0.15 D (preferably around 0.097 D). Fiber density should be around 90,000-110,000 fibers per square inch (preferably around 96,768) and loop density should be around 1,000,000-1,500,000 loops per square inch (preferably around 1,382,400). In some embodiments hooks and fibers of the inventive subject matter can be made from materials like nylon and polyester.

The end result is a fabric that can be attached to itself in such a way that gives rise to high sheer strength with low breakaway strength. In preferred embodiments, the sheer strength of a fabric that has been attached to itself (e.g., a top side couples with a bottom side as with a wrap) is greater than the breakaway strength. Sheer strength refers to the strength of a fabric coupled with itself and pulled along the length of the strip of fabric, while breakaway strength refers to the forces required to pull a piece of fabric off of itself after it has been attached (force largely perpendicular to the sheer direction).

Sheer holding strengths of wraps of the inventive subject matter when coupled together (e.g., when the fabric side is coupled with a loop side) can range from 0.01 to 1.2 kgf/cm² (±5%). In other words, with that amount of sheer force applied, the wrap will still hold without breaking away. For example, a thin knit fabric with fiber and loop bonding that is made from 97% polyester and 3% OP yield a holding strength of at least 0.04 kgf/cm². In another example, a thin knit fabric made from single layers of fiber and loop that are formed from 100% polyester yield a holding strength of at least 0.02 kgf/cm² (±5%). Different wraps can be created to exhibit different sheer holding strength characteristics.

Fabrics used to create wraps of the inventive subject matter are soft to the touch on both sides and have elastic qualities-a significant advantage over ordinary hook and loop, which can be stiff, scratchy, and flexible but not stretchable. Thus, ordinary hook and loop materials (e.g., Velcro) cannot be substituted for fabrics of the inventive subject matter. Fabrics of the inventive subject matter, as discussed above, have one side with fibers and another side with loops, where the fibers can interact with the loops to create a “sticky” effect. Because those fibers and loops are made using threads having such low linear density and because the density of fibers and loops is so high, the fabrics feel soft to the touch.

Wrap 100 is formed as a thin (e.g., 0.05 mm to 7 mm thickness) and wide (e.g., 1-20 cm) tape such that it can be stretched when used. Different wraps can have different elastic properties, and because those elastic properties are known and consistent for a particular wrap material, lines marked on a wrap of the inventive subject matter can be used to apply a known amount of pressure when used on an individual's body. FIG. 2 shows an example of how wrap 100 can be applied to a user's lower leg.

Wraps of the inventive subject matter can have different characteristics depending on embodiment. Low-stretch bandages, also known as short-stretch bandages, are compression bandages that—in general—cannot stretch more than 100% of their original length. Fabrics that make low-stretch bandages have relatively high stiffnesses and are used to create a tight containment against which a wearer's muscles contract. In contrast, ordinary wraps like Ace Bandages are long-stretch wraps. Long-stretch wraps can stretch beyond 100% of their original length.

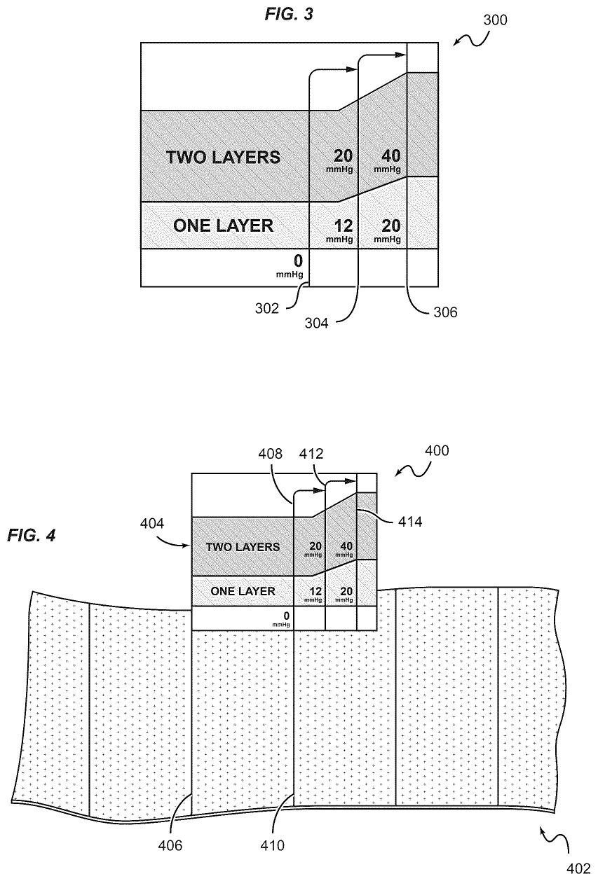

In some embodiments, different wrap styles can be combined. For example, a long-stretch fabric can be used as a base, with a short-stretch wrap over the top. In such a configuration, a thin short-stretch wrap can be used over the top of a long-stretch wrap because both wraps together would apply a suitable amount of pressure. If a thicker short-stretch wrap is used over a long-stretch wrap, the wearer may experience too much pressure. Stretching keys, such as the one shown and described in FIG. 3 et seq., can be created to account for multiple fabric materials and multiple layers.

Because wrap 100 has known material properties, forming wrap 100 to a specific thickness and a specific width enables wrap 100 to apply a known amount of pressure when stretched prescribed amounts when applied to a user. This is gives rise to the importance of lines 102 when used in association with a key as shown in FIG. 3. Key 300 should printed on a firm, but flexible material, such as a laminated paper or a plastic. The material should be flexible enough to bend around a user's body while still being stiff enough to prevent creasing or overlapping of its material so that key 300 can be used to measure how much a wrap is stretched.

Key 300 provides information to facilitate use of a wrap and should be included as a component of a system. Key 300 shows a first line 302 to indicate no pressure, a second line 304 to indicate a first pressure, and a third line 306 to indicate a third pressure. The first and second pressures correspond to wrapping the wrap around a user's body part to create one layer. Key 300 also indicates how much pressure could be applied by creating a second layer (e.g., by wrapping the wrap around the body part a second time). The example given indicates the first pressure is 12 mmHg and the second pressure is 20 mmHg, while a third pressure (two layers to the second line) is 20 mmHg and a fourth pressure (two layers to the third line) is 40 mmHg. The amount of pressure created by a given system depends on the material properties of a wrap, the dimensions of the wrap, the distance between the lines on the wrap, and the distance between the lines on a key.

FIGS. 4-6 show how to use a key 400 with a wrap 402. FIG. 4 shows key 400 resting against a length of wrap 402 having lines spaced at regular intervals. Key 400 has a key base line 404 (which is coincident with an edge of key 400, as drawn) that is aligned with a first wrap line 406. A second key line 408 is shown aligned with a second wrap line 410. In this configuration, no pressure is applied because wrap 402 is not stretched at all.

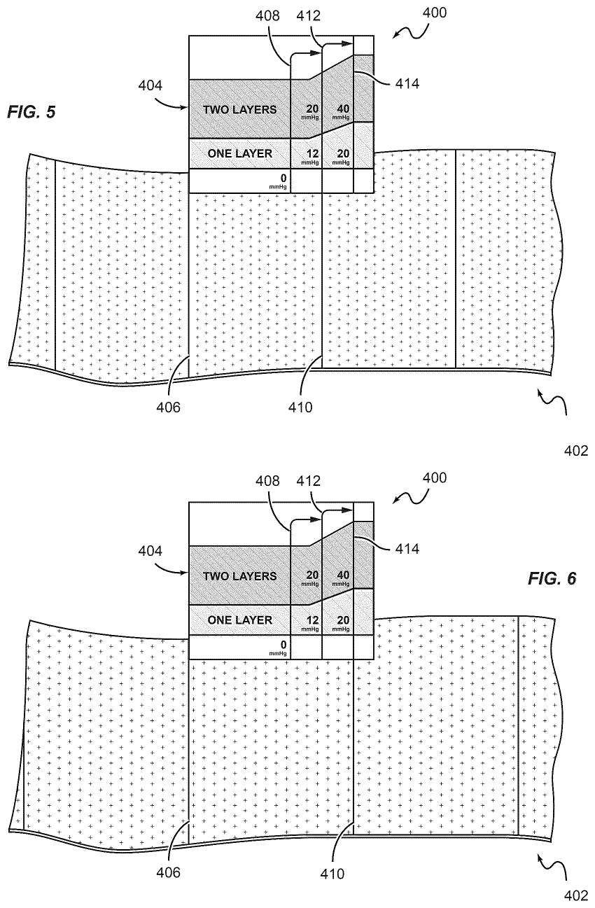

FIG. 5 shows wrap 402 stretched slightly. Key base line 404 and first wrap line 406 are still aligned, but second wrap line 410 is now aligned with third key line 412. As shown in FIG. 5, this gives rise to a pressure of 12 mmHg when there is one layer of wrap 402 and a pressure of 20 mmHg when there are two layers of wrap 402.

FIG. 6 shows wrap 402 stretched even more. Key base line 404 and first wrap line 406 are still aligned, but second wrap line 410 is now aligned with fourth key line 414. As shown in FIG. 6, this gives rise to a pressure of 20 mmHg when there is one layer of wrap 402 and a pressure of 40 mmHg when there are two layers of wrap 402.

Embodiments of the inventive subject matter are widely applicable in sports, physical therapy, medicine and so on. By creating a wrap that can be used to apply a specific and known amount of pressure, medical practitioners and the like can more effectively apply a wrap. This can be beneficial, for example, with lymphedema patients who can benefit from wraps applied to affected limbs, and where applying such a wrap to a specified pressure can enhance the benefit of the wrap. Wraps can also be used by athletes who want to apply a known pressure to a limb or muscle group to treat an injury.

Embodiments of the inventive subject matter can be used in a variety of different contexts. For example, a wrap can be used with splinting materials such as wood or plastic. A splint is a stiff member that can be used to create structural rigidity, and so it is frequently elongated, stiff, and having a length according to its intended use on the body. A split can thus provide rigidity in situations where movement can cause injury or harm, such as if a bone is broken or a joint is injured. By using a wrap of the inventive subject matter in association with a splint, a person can be braced with the splint with the benefit of applying a known amount of compression to the person's body.

Wraps can also be used in association with a fabric base, such as a sock. For example, in some embodiments, after a user puts on a sock, they can apply a wrap of the inventive subject matter over the sock to create the base for an ankle brace boot. A person can alternatively apply a wrap of the inventive subject matter first and then put on a sock over the wrap to prevent the wrap from being impacted (e.g., peeled apart or disconnected in any way) when a user puts on a boot or shoe.

Wraps of the inventive subject matter can also be used to apply pressure to other garments to prevent or minimize slipping. For example, a wrap can be used with arm sleeves that cyclists wear to stay warm (these sleeves are not attached to a shirt-instead they are just sleeves that can be worn on their own). By applying a wrap to the sleeve, the wrap can prevent the sleeve from slipping.

Wraps of the inventive subject matter can be used to apply warming/cooling packs, as well. Warmth and cold are often used in sports medicine to treat inflammation or injury, or even just to improve recovery after exercise. To maximize effectiveness, warmth and cold should be applied to specific parts of the body, and a wrap of the inventive subject matter can be used to apply the warmth/cold at a known pressure, combining both therapeutic effects.

Embodiments can also be used in association with gauze or other medical bandages. In many cases, when gauze or medical bandage is applied, it can be beneficial to also apply a known amount of pressure at the same time. This can help slow bleeding, for example.

A key, as described above, may not always be available, or a key may not provide pressure increments that are needed. Custom pressures can be created by using a pressure sensor and a measuring tape to measure how far a wrap needs to be stretched to create a specific pressure. Once the amount of stretch is known, it can be repeated using the measuring tape as a makeshift key.

In some embodiments, a wrap of the inventive subject matter can incorporate a camo pattern. By incorporating a camo pattern, a wrap can be used in military contexts, e.g., over a person's battle dress uniform, without compromising the person's camouflage and without impacting a person's infrared signature. This allows for in-field, over-garment treatment to ensure speed of application while maintaining integrity of a camo pattern.

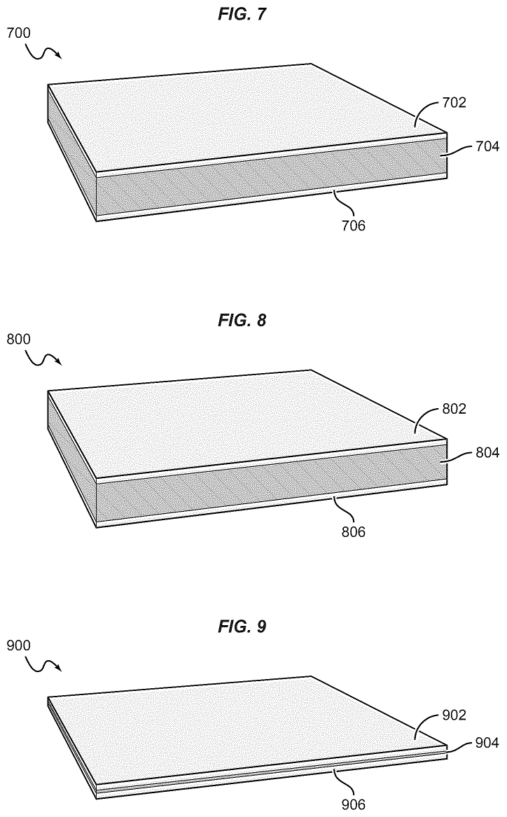

Different cores can also be used to bring about different material properties for wraps of the inventive subject matter. FIGS. 7-9 are included to show several different cores that can be used by showing them in small portions of wraps of the inventive subject matter.

FIG. 7 shows a wrap having an EVA (ethylene vinyl acetate) foam core. An advantage of using an EVA foam core is that it adds to the wrap's overall hardness (and, accordingly, stiffness), which can improve performance of the wrap when used with accessories like splints. The EVA core adds hardness without sacrificing too much in weight, creating a lightweight wrap that works well with splints and the like. Thus, wrap 700 features a top layer 702, an EVA foam core layer 704, and a bottom layer 706. The top and bottom layers are configured such that either a fiber side or a loop side of a wrap material as described above is facing away from the EVA foam core layer 704. Thus, wrap 700 can still be wrapped around a person's body or body part in a way that it sticks to itself (e.g., the fiber side of the wrap interacts with the loop side when used as a wrap).

FIG. 8 shows a wrap having a PU (polyurethane) foam core. Thus, wrap 800 includes a top layer 802, a PU foam core layer 804, and a bottom layer 806. PU foam cores can add rigidity to wrap 800 that can be useful when covering areas where the body has extreme contours that may prevent folding of wrap 800. For example, when wrapping around the end of a limb with lymphedema or while transitioning planes, the structure of the product keeps the curve of the wrap as it goes around the body part more uniform. This can also help create more constant pressure for a user during decompression (i.e., pushing fluid out of the region) as the PU foam core can introduce additional spring force. PU foam cores can also improve breathability as compared to EVA foam cores. The top and bottom layers 802 and 806 are configured such that either a fiber side or a loop side of a wrap material as described above is facing away from the PU foam core layer 804. Thus, wrap 800 can still be wrapped around a person's body or body part in a way that it sticks to itself (e.g., the fiber side of the wrap interacts with the loop side when used as a wrap).

In another embodiment, a TPU (thermoplastic polyurethane) film can be implemented in a middle layer of a wrap. Wrap 900 features a top layer 902, a TPU film 904, and a bottom layer 906. TPU film can be used to, e.g., form a wrap of the inventive subject matter by bonding top layer 902 to bottom layer 906. This is how most wraps of the inventive subject matter are formed, having a fiber surface and a loop surface bonded by a TPU film. By changing the material properties of the TPU film 904, the resulting wrap 900 can have different rigidities, elasticities, and compression ratios. These material characteristics can be engineered through different types of films such as a combination that has low melt layer, then a high melt layer, then another low melt layer. The low melt layer acts as the glue that bonds the TPU film to the fiber and loop layers, while the high melt layer is used for its material properties, such as its elasticity.

In still further embodiments a spacer mesh core can be used. Such a wrap would appear similar to the wraps shown in FIGS. 7 and 8, having a top layer, a spacer mesh core layer, and a bottom layer. A spacer mesh core is a material that is porous, so that it can add bulk to a wrap through thickness while keeping improved breathability beyond the capability of, e.g., PU foam. Alternatives to spacer mesh cores can also be used, such as needle punch fabric. As with foam cores, needle punch fabric can add bulk and thickness. An advantage of needle punch fabric over foam is that needle punch fabrics are typically less compressible. This is an advantage in scenarios where more immobilization is needed.

In still further embodiments, wraps of the inventive subject matter can themselves be configured as bandages (as opposed to being a wrap that goes over a bandage). For a wrap to function as a bandage, the wrap can have a coating applied to improve its characteristics as a bandage. For example, in a wrap that comprises a top layer, a bottom layer, and a core, a loop side of a wrap can be treated with a non-stick coating to help prevent it from sticking to a wound, the core can be formed from an absorbent material such as thick textile or hydrogel, and the fabric side of the wrap can comprise a waterproof coating or be made from a fabric having waterproof qualities.

In another embodiment, the loop side of the warp can be treated with a non-stick coating to help prevent it from sticking to the wound and the core can be made using an absorbent material such as thick textile or hydrogel. The outer layer would then be the fabric side that is configured to couple with the loop side.

Thus, specific systems and methods directed to compression wraps have been disclosed. It should be apparent, however, to those skilled in the art that many more modifications besides those already described are possible without departing from the inventive concepts in this application. The inventive subject matter, therefore, is not to be restricted except in the spirit of the disclosure. Moreover, in interpreting the disclosure all terms should be interpreted in the broadest possible manner consistent with the context. In particular the terms “comprises” and “comprising” should be interpreted as referring to the elements, components, or steps in a non-exclusive manner, indicating that the referenced elements, components, or steps can be present, or utilized, or combined with other elements, components, or steps that are not expressly referenced.

What is claimed is:

1. A compression wrap system, comprising:
an elongated fabric material having a first side, a second side, and a series of equally spaced lines across the elongated fabric;
the first side comprising loops and the second side comprising fibers;
wherein the first side is configured to stick to the second side;
a key having a first line and a second line; and
wherein, when using the elongated fabric as a wrap, aligning the first line of the key with a first equally spaced line on the elongated fabric and stretching the elongated fabric such that a second equally spaced line is aligned with the second line of the key results in a known pressure being applied by the wrap.

2. The compression wrap system of claim 1, wherein the key comprises an edge, and the edge coincides with the first line.

3. The compression wrap system of claim 1, wherein the loops have a linear mass density of between 0.04 deniers (D) and 0.15 D.

4. The compression wrap system of claim 1, wherein the loops on the first side have a density of between 1,000,000 and 1,500,000 loops per square inch.

5. The compression wrap system of claim 1, wherein the fibers have a linear mass density of between 1 D and 5 D.

6. The compression wrap system of claim 1, wherein the fibers on the second side have a density of between 90,000 and 110,000 fibers per square inch.

7. A compression wrap system, comprising:
a wrap comprising an elongated fabric material;
the wrap having a first side, a second side, and a series of equally spaced lines across a width of the wrap;
the first side comprising loops and the second side comprising fibers;
wherein the loops of the first side are configured to stick to the fibers of the second side;
wherein, upon applying a fixed amount of stretch to the wrap when the wrap is wrapped around an object, a known amount of pressure is applied; and
wherein the fixed amount of stretch is measured using the equally spaced lines.

8. The compression wrap system of claim 7, further comprising a key having a first line and a second line, wherein the key is used to measure the fixed amount of stretch by aligning the first line with a first equally spaced line and the second line with a second equally spaced line.

9. The compression wrap system of claim 8, wherein the key comprises an edge, and the edge coincides with the first line.

10. The compression wrap system of claim 7, wherein the loops have a linear mass density of between 0.04 deniers (D) and 0.15 D.

11. The compression wrap system of claim 7, wherein the loops on the first side have a density of between 1,000,000 and 1,500,000 loops per square inch.

12. The compression wrap system of claim 7, wherein the fibers have a linear mass density of between 1 D and 5 D.

13. The compression wrap system of claim 7, wherein the fibers on the second side have a density of between 90,000 and 110,000 fibers per square inch.
